(19) United States
(12) Patent Application Publication
(10) Pub. No.: US 2016/0270715 A1
(43) Pub. Date: Sep. 22, 2016

(54) Methods of Treating Wrinkles, Developing Wrinkle Treatments And Evaluating Treatment Efficacy, Based On Newly Discovered Similarities Between Wrinkles And Skin Wounds

(71) Applicant: ELC Management LLC, New York, NY (US)

(72) Inventors: Thomas Mammone, Farmingdale, NY (US); Vasile Ionita-Manzatu, Bethpage, NY (US); Geoffrey Hawkins, Yardley, PA (US); Glen Rein, Northport, NY (US)

(21) Appl. No.: 14/661,218

(22) Filed: Mar. 18, 2015

(51) Int. Cl. A61B 5/00; A61B 5/053

(52) U.S. Cl. CPC A61B 5/442; A61B 5/0531; A61B 5/0051

(57) ABSTRACT

Disclosed are methods of measuring an endogenous wrinkle electric field and methods of characterizing topical applications, or other treatments, in terms of their effect on the endogenous electric field of wrinkles. Also included, are methods of developing topical applications, or other treatments that have a desired effect on the endogenous electric field of a wrinkle. The methods disclosed herein, are based on a new model of wrinkle physiology that exploits previously unknown similarities between skin wounds and wrinkles.

### FIELD OF THE INVENTION

The invention is in the field of skin treatment, cosmetics and dermatologics. In particular, the invention provides novel methods for evaluating skin wrinkles, and novel approaches to skin care, by characterizing endogenous electric fields in the skin.

### BACKGROUND OF THE INVENTION

The dermis and epidermis are the two main layers of human skin. Over most of the body, the epidermis is about 0.1 mm thick, increasing up to about 2 mm thick on the palms and soles. Depending on location, the epidermis is subdivided into four or five strata. Each stratum is mainly composed of multiple layers of continuous sheets of stratified squamous epithelial cells. About 90% of epidermal cells are keratinocytes. From stratum to stratum, the shape and composition of these cells changes. Over an approximate one month period, epithelial cells “born” in the basale, migrate up the strata, changing their shape and composition as they move toward the skin surface, where they are eventually sloughed away. Deep to superficial, the five strata are: the germinativum (or basale: a single layer of cells; here, stem cells divide to produce keratinocytes that populate the upper layers of the epidermis); the spinosum (about 8 to 10 layers of tightly packed cells); the granulosum (about 3 to 5 layers of dead and dying cells); the lucidum (not found in hairy skin: 3 to 5 layers of flat, dead cells); the corneum (15 to 30 layers of flat, dead, keratinized cells).

The term “apical surface” refers to a free surface of an epithelial cell, which has no contact with another cell. For example, the skin outer surface is the apical side of the top layer of epithelial cells. The term “basal surface” refers to those epithelia that are attached to a basement membrane. Thus, over an approximate one month period, epidermal cells migrate from the basal surface to the apical surface. For directional or spatial reference, we also speak of the apical side of any epithelial cell, and mean the side closer to the free surface (i.e. outer skin surface). Also, the basal side of an epithelial cell means the side closer to the basement membrane. Depending on the shape of the cell, we can also identify a lateral surface between the basal and apical surfaces.

Despite their constant migration, epidermal cells are tightly packed together to give the tissue its protective and barrier properties. Adjacent cells do not contact each other along the entire length of their membrane perimeter. Rather, there is a gap between cells, except at well defined sites, where a cell junction occurs. Within the gaps, is an extracellular fluid. There are several types of cell junctions, of which “tight”, “adherens” and “desmosomes” are of interest in the epidermis. In general, adjacent epidermal cells form tight junctions on the lateral membrane, near the apical surface. As a result, the apical cell membrane is functionally different from the basal and lateral cell membranes (hereafter, basolateral membrane) and passage of materials is restricted through the tight junction. Consequently, the extracellular environment of the apical membrane is generally different from that of the basal membrane.

The epidermis is securely bonded to the dermis at the basement membrane. The more superficial layer of the dermis comprises papillae that project into the overlying epidermis. The deeper layer of the dermis is a connective tissue matrix, comprised of collagen and elastin fiber networks.

### Electric Potential Across Healthy Epidermal Cells and the Transepithelial Potential

As noted above, within a single epidermal cell, the basolateral membrane is functionally different from, and to some extent isolated from, the apical cell membrane. For example, channels through which Na⁺ can passively diffuse through the epithelial membrane, are concentrated on the apical side of the cell, while K⁺ channels are concentrated on the basal and lateral sides of the cell. Also, within normal human epithelial cells, Na⁺/K⁺ ATP-ase operates on the basolateral membrane to actively move Na⁺ out of the cell, and to increases the concentration of K⁺ inside the cell. This is not the case at the apical membrane. As a result, the interior of each living, healthy epithelial cell is electrically negative, compared to the environment just outside the cell.

Thus, while Na⁺/K⁺ ATP-ase increases the concentration of Na⁺ just outside the basolateral membrane, there is a passive influx of Na⁺ through the channels on the apical membrane. Also, as Na⁺/K⁺ ATP-ase actively raises the concentration of K⁺ inside the cell, there is an efflux of K⁺ out the K⁺ channels on the basal membrane. As a result, there is an increasing concentration of positive ions just outside the basolateral membrane and a relative depletion of positive ions just outside the apical membrane. In response to this, there is a tendency for ions to flow in the extracellular fluid, from the basolateral membrane toward the apical membrane, in the gap between epidermal cells (the paracellular pathway). This flow of ions is hindered (but not prohibited), by the cell junctions that exist along the lateral membrane. Thus, a concentration of positive charge exists behind the junction (at the deeper layers), and a positive charge depletion exists ahead of the junction (at the more superficial layers). This charge separation gives rise to a charge potential, known as the transepithelial potential. The potential is relatively negative on the apical side of the junction.

Human epidermis is composed of multiple layers of epithelial cells (i.e. the cells are stratified). A closed loop electric current is associated with each living cell. In each layer of cells, the individual cell currents move superficial to deep within the cell, and deep to superficial outside the cell. Because the currents are aligned, normal, healthy epidermis exhibits a characteristic net electric potential that measures about 20-50 mV between the top and the bottom of the epidermis, with the bottom of the epidermis being more positive than the top. The composite effect of these individual, aligned potentials, has been called the “skin battery”.

For the most part, the ionic current in healthy epidermis, moves between superficial and deep, and does not have a significant net component in a lateral direction (i.e. parallel to the skin surface). Thus the charge potential of the healthy skin battery is aligned approximately perpendicularly to the skin surface. This changes when the epidermis is broken.

### Electric Potential Across Wounded Epidermis

A wound in the skin may be confined to the epidermis (a scrape, for example) or may extend down into the dermis (a laceration, for example). In either case, the wound site is depleted of epidermal cells. In repair of an epidermal wound, epidermal cells near the perimeter of the wound, migrate into the wound site until the wound is resurfaced. The migrated cells are replaced by new cells from the basal strata. Typical healing times may be 24-48 hours. A purely epidermal wound involves little or no inflammatory response and no scarring. In contrast, a deeper wound, in the dermis and/or below, involves the four phases known as inflammatory phase, migratory phase, proliferative phase and maturation phase. The healing time is considerably longer than an epidermal wound and scarring often results.

As noted above, healthy epidermis exhibits a characteristic net charge potential that measures about 20-50 mV between the top and the bottom of the epidermis. Between any two points on the epidermis, we can talk about a difference in potential. It has long been known that the electric potential between two points on the skin, is changed by a wound in the skin between those two points. It is further known that, as the wound heals, the electric potential tends to return to pre-wound levels. Also, it has been observed that a charge current exits or moves away from the site of skin wound.

In terms of the skin battery, a break in the epidermis disrupts the normal current loop and establishes a wound current and wound electric field. A break in the epidermis creates a low resistance path for extracellular ions. Driven by the transepithelial potential in the epidermis immediately adjacent to the wound, new current loops are established. Nearer the skin surface, just below the stratum corneum, positive ions flow laterally, away from the site of the wound for a short distance; then, the current turns and moves to deeper layers of the epidermis (into the stratum spinosum, for example), eventually turning again to flow laterally toward the wound site, along the basal side of the epidermis. Finally, the circuit ascends to where it started, forming a closed loop.

The loop has two lateral components with opposite polarity; the more superficial lateral electric field is positive at the wound and negative away from the wound; the deeper lateral electric field is negative at the wound and positive away from the wound. One characteristic of skin adjacent to a wound site is a positive polarity toward the more superficial layers of the epidermis, and a negative polarity toward the deeper layers. This is the opposite of the polarity of the transepithelial potential of healthy skin and it is the lateral component of the wound electric field that directs the migration of nearby epidermal cells, during the healing process.

### Measurements of the Endogenous Skin Electric Field

The first measurements of ionic currents exiting the site of a skin wound, were reported in 1843, by DuBois-Reymond, who measured the currents with a galvanometer (see, DuBois-Reymond, E. (1843). Vorlaufiger abrifs einer untersuchung uber den sogenannten froschstrom and die electomotorischen fische. Ann. Phys. U. Chem. 58, 1.). In 1980, exit wound electric currents were confirmed using a vibrating probe technique (see, Illingworth, C. M. and Barker, A. T. (1980) Measurement of electrical currents emerging during the regeneration of amputated fingertips in children. Clin. Phys. Physiol. Meas. 1, 87-89). Vibrating probe techniques have been used to measure ionic currents in the liquid media, in and around cells and tissues (see Jaffe and Nuccitelli, (1974) An ultra sensitive vibrating probe for measuring extracellular currents. J. Cell Biol. 63, 614-628).

It was long understood that wound exit currents give rise to an electric field, in and around the skin that borders the wound (hereinafter, wound electric field). The wound electric field has been measured, for example, in the skin of guinea pigs (see Barker, A.T., Jaffe, L.F., and Vanable, J.W., Jr. (1982). The glabrous epidermis of cavies contains a powerful battery. Am. J. Physiol. 242, R358-R366; also see Jaffe, L. F. and Vanable, J. W. (1984). Electric fields and wound healing. Clinical Dermatology 2, 34-44). The wound electric field has also been measured in newts (see McGinnis, M. E. and Vanable, J. W., Jr. (1986). Electrical fields in Notophthalmus viridescens limb stumps. Dev. Biol. 116, 184-193). However, for a long time, measurements in human skin were hindered by the difficulty of using standard microelectrode technology in human wounds. Part of the difficulty of measuring electric fields in skin, lay in the fact that the probe had to contact the skin.

Recently, a new generation of non-contact vibrating probe devices have been developed that can measure electric fields of the skin, while the probe remains in the air, instead of within inter- and intracellular fluid. A commercial version of this non-contact imaging instrumentation is known as the Dermacorder® (available through BioElectroMed Corp. Burlingame, Calif.). The Dermacorder® has been used successfully to quantify the electric field associated with certain skin lesions. The Dermacorder® is capable of non-invasive measurement of bioelectric fields in humans, leading to a mapping of the electric field. The Dermacorder® does not require the electrodes to contact the skin, at the wound site. Recently, the electric field pattern surrounding skin wounds in mice and humans, has been reported (Nuccitelli R. et al., (2008) Imaging the electric field associated with mouse and human skin wounds. Wound Rep. Reg. 16, 432-441). Wounds in humans were found to generate electric fields on the order of 80 mV/mm. This is compared to 20-50 mV for healthy skin. Thus, for the first time, relying on a non-invasive technique, it was possible to map or image the electric field lines associated with human skin wounds.

Following a wound, the electric field, generated by the wound's exit current, initiates a wound healing process. The electric field does this by triggering a migration of keratinocytes toward the wounded region, via galvanotaxis. It is believed that the shape and character of the wound electric field correlates to the outcome of wound healing. For example, “abnormal” electric fields may exhibit lower healing rates. If true, this would suggest that treatments that alter the electric field at a wound site (hereafter, “wound electric fields”), may have an effect on the outcome of healing. Thus, a wound electric field imaging technique, such as the Dermacorder®, would make possible the diagnosis and treatment of wounds, including chronic, non-resolving wounds. Such an imaging technique would be able to differentiate between normal wound electric fields and abnormal wound electric fields, thus suggesting treatment for non-resolving or slowly resolving lesions, for example chronic ulcers and bedsores. According to Reid et al., “There is increasing evidence that electric currents may play an important role in development and healing processes in vertebrate skin . . . . These results also suggest a possible new approach of using pharmacological agents to enhance or decrease endogenous electric fields in various clinical situations where physically applying electrodes is difficult or impossible” (“Wound healing in rat cornea: the role of electric currents” The FASEB Journal vol. 19 March 2005, pp. 379-386).

### Skin Wrinkles

Wrinkled skin that is characteristic of aging is partly the result of a degeneration of the collagen and elastin matrix that make up the deeper layer of the dermis. With age, the concentration of collagen fibers in the dermis decreases and the fibers themselves become more brittle. As a result, the collagen matrix fails to hold its shape. At the same time, elastin fibers lose some of their elasticity, so that the skin's ability to return to shape after it has been stretched is compromised. The lines and wrinkles characteristic of aging skin are the result.

There are many differences between a skin wrinkle and a wound. In general, wrinkles do not involve breaks in the epidermis or dermis, like a wound. Also, it has been observed, that the epidermis of ageing skin gets thinner. Moreover, it has been observed that the living layers of the epidermis are generally thinner near the bottom of a skin wrinkle, than at the sides of the wrinkle. This loss of skin thickness with age is associated with an observed reduction in the number of cell layers in one or more strata of the epidermis. Thus, at the bottom of a wrinkle, the number of cell layers of stratum spinosum may be less than unwrinkled skin. Furthermore, with age, the dermal-epidermal junction becomes flatter and, at the bottom of a wrinkle, the dermal-epidermal junction is losing collagen faster than the sides of the wrinkle. (see Contet-Andonneau, et al, (1999) “A histological study of human wrinkle structures: comparison between sun-exposed area of the face, with or without wrinkles, ad sun-protected areas” British Journal of Dermatology vol. 140, pp. 1038-1047). Other differences between a wrinkle and wound include: cell migration into the site of a wrinkle is not known to occur, as it does at a wound, and the four phase inflammatory response, characteristic of a deep wound, are not associated with a wrinkle. Certainly, overall, wrinkled skin seems to be much more similar to normal, unwrinkled skin than it does to a wound.

Thus, it was heretofore unexpected, that a wrinkle would have an electric field very different from unwrinkled skin. It was also unexpected that the electric field of a wrinkle would have significant similarities with the electric field of a wound. Also, heretofore, a vibrating probe device had not been used to measure or image the endogenous electric field of a wrinkle (hereafter, “wrinkle electric field”). Also, treatment of wrinkles based on that type of measurement is unknown in the prior art.

At this point, a distinction needs to be made between epidermal electric fields, which may span a wrinkle in the skin, and those electric fields that arise specifically because of the wrinkle. In the long history of measuring electric fields associated with skin, it seems likely that the area across which an electric potential was measured, included a wrinkle. But this is different from measuring the electric field that is endogenous to the wrinkle, that exists because of the wrinkle or that exists as a precursor to a wrinkle. Also, it is true that skin wrinkles have been subjected to applied electric fields, but this is different from the targeted methods of the present invention that expose a wrinkle to one or more specific electric fields based on the magnitude and polarity of the wrinkle electric field.

It is also important to distinguish the methods of the present invention from wrinkle treatments involving laser resurfacing and pulsed light technology. The application of electromagnetic fields to the skin for ablating the skin is different from the present invention, with methods do not ablate the skin.

It is also important to distinguish the methods of the present invention from “imaging” methods discussed in U.S. Pat. No. 6,944,491 and related applications. The '491 patent claims to disclose a method of acquiring an image of a zone of the skin or hair, in order to determine certain parameters of the zone and/or to make a diagnosis, wherein the image is acquired by means comprising at least one non-optical sensor. According to the '491 reference, the non-optical sensor may have an active surface that is sensitive to at least one electrical magnitude, e.g. electrical charge. Also, the sensor may be a non-contact sensor and has a resolution between 10 and 100 μm.

What must be understood, however, is that the '491 reference does not describe any particular data acquisition device or imaging method. Nor does it describe any particular variable measured by the device. The focus of the '491 reference is processing the data after the data has been acquired. Thus, the reference says:
“The image processing that is performed seeks, for example, to determine one or more magnitudes that are characteristic of the microrelief of the skin so as to be able to deduce information therefrom about the state of the skin . . . ” (col. 3, lines 50-54)

Thus, the '491 patent simply says that if known data acquisition methods are used to acquire data, then that data can be reduced to yield information on the state of the skin. Examples of the type of information that may be gleaned from the acquired data include:
“ . . . with the resulting information relating, for example, to the probable concentration in the skin of macromolecules forming the extra-cellular matrix of conjunctive tissue, i.e. collagen, elastin, proteoglycans, and glycoproteins, and/or concerning the orientations of collagen bundles relative to the axis of an arm, amongst other things.” (col. 3, lines 50-62)
“The image processing which is performed also seeks to provide information concerning the density of skin lines and more particularly the anisotropy coefficient of line density, i.e. the ratio of line density in a first direction to line density in a second direction, substantially perpendicular to the first.” (col. 3, lines 63-67)
“The processing performed on the image can also seek to determine the number and the size of pores in the skin, or indeed the size and/or the density of the plateaus as defined by the lines.” (col. 4, lines 1-4)
“The processing performed on the image can also serve to quantify and/or characterize the wrinkles present in the skin and to give information concerning pilosity.” (col. 4, lines 5-7)
“The person skilled in the art will readily understand that such an image can be subjected to processing making it possible to determine the surface density of the lines L and the anisotropy coefficient of this density, i.e. the ratio of the density of lines L in an X direction to the density of lines L in a Y direction perpendicular thereto.” (col. 6, lines 53-58)

No where does the '491 reference disclose which parameters need to measured, in order to have data that can yield these types of information. No where does the reference disclose actual methods of acquiring data. No where does the reference disclose actual methods of processing the acquired data. The reference merely suggests that known methods of skin measurement, combined with known methods of data processing, can yield expected information about the state of the skin, and that that information can be used to decide a course of treatment or evaluate the effectiveness of a course of treatment. Which is to say that the '941 patent (as exemplified in claims 1, 28, 33, 34, 45 and 54) does not disclose anything that was not already known. For example, concerning wrinkles, the complete disclosure of the '491 reference is:
“The processing performed on the image can also serve to quantify and/or characterize the wrinkles present in the skin . . . ” (col. 4, lines 5-7) and
“When contact is necessary and the image delivered by the sensor changes with changing pressure exerted by the sensor on the observed zone, it is advantageous, as shown in FIG. 4, to use a suitable detector 8 on which the sensor 4 is mounted to measure the contact pressure associated with each image, so as to extract 3D information from the way images vary with pressure. This makes it possible with suitable data processing to determine the profile of a wrinkle, for example.” (col. 7, lines 32-40)

The '491 reference does not disclose measuring the electric field of a wrinkle. It does not disclose measuring the electrical polarity of the epidermis in the vicinity of a wrinkle. It does not disclose using a non-contact vibrating probe technique to measure electric properties of wrinkles. The '491 reference does not disclose a new approach to wrinkle treatment that involves considering a wrinkle to be a wound in the skin.

Methods of treating wrinkles with electric current are known. One example is disclosed in U.S. Pat. No. 6,684,107. Generally, an electric current is passed between points of the skin in the vicinity of a wrinkle. In the case of U.S. Pat. No. 6,684,107, a 10-40 micro-amp current is passed through the surface of the wrinkle into the underlying dermal layer. This technique is alleged to “reduce” the wrinkle. Applicants point out that passing a current through a wrinkle in the manner described in the '107 reference is different from the methods of present invention for applying an electric field to a wrinkle. The '107 reference does not disclose measuring the electric field of a wrinkle. It does not disclose measuring the electrical polarity of the epidermis in the vicinity of a wrinkle. It does not disclose using a non-contact vibrating probe technique to measure electric properties of wrinkles.

To the best of the applicant's knowledge, no one has measured the electric field strength specifically associated with a skin wrinkle, either with a Dermacorder®, or with any other device. No one has measured an electric potential that is specifically associated with or that specifically arises from the presence of a wrinkle. No one has used this information to select an electric field to apply to a skin wrinkle, with the aim of altering the wrinkle. Thus, methods of predicting wrinkles based on electric field readings of the skin are new. Corrective and preventive wrinkle treatments based on altering the electric field properties of skin wrinkles are new. Evaluating the efficacy of skin wrinkle treatments based on the electric field of skin wrinkles is new.

### OBJECTS OF THE INVENTION

A main object of the invention is to provide methods of characterizing the electric field that arises specifically from a skin wrinkle.

Another object of the invention is to provide methods of treating or preventing wrinkles, based on electric field properties of wrinkles.

Another object of the invention is to provide methods of characterizing the progression of a skin wrinkle, based on electric field properties of wrinkles.

Another object of the invention is to provide methods of evaluating the efficacy of a skin wrinkle treatment, based on the electric field properties wrinkles.

Another object of the invention is to provide methods of comparing the efficacy of two skin wrinkle treatments, based on electric field properties of wrinkles.

Another object of the invention is to provide methods of formulating topical wrinkle treatment products, based on the electric field properties wrinkles.

### SUMMARY OF THE INVENTION

The invention lies, in part, in methods of obtaining useful characterizations of skin wrinkles based on endogenous wrinkle electric fields. Measurements of the electric fields of skin wrinkles are done with one or more devices capable of such measurements, the measurements being done in a manner that is capable of yielding useful characterizations of skin and wrinkle behavior. The invention includes methods of characterizing topical applications, or other treatments, in terms of their effect on the endogenous electric field of wrinkles. Also included, are methods of developing topical applications, or other treatments that have a desired effect on the endogenous electric field of a wrinkle.

### DETAILED DESCRIPTION

Throughout the specification, the term “comprises” or variations, thereof, means that an article or method is not limited to the items specifically recited.

As discussed above, a skin wrinkle is not a wound. Therefore, there was no reason to expect the existence of an electric field endogenous to a wrinkle, having significant similarities to a wound electric field and significant differences from the electric field of normal or healthy, unwrinkled skin. Once we postulated the existence of electric fields that are endogenous to wrinkles, there remained the task of demonstrating their existence. To this end, we made a study of five male subjects and one female subject, according to the following protocol.
1. Obtain signed informed consent forms for each participant.
2. Have subject lie down on examining table, and immobilize head in a restraint system.
3. Thoroughly clean forehead with sterile alcohol wipe.
4. Mark three wrinkles with fine-tipped marker.
5. Apply ground electrode (3M Red Dot Ag/AgCl).
6. Use Dermacorder® to scan each wrinkle. Wrinkles are scanned at the wrinkle center and 3 mm on either side. The off wrinkle values are averaged and subtracted from the values measured in the center of a wrinkle.
7. Use Surface Probe coated with conductive gel (Signa Gel, Parker Laboratories, N.J.) to measure the surface potential of the stratum corneum in center of wrinkle, raise electrode and lower for a second reading in the same location.
8. Move the electrode 500 μm away from center and measure the potential there twice.
9. Move an additional 500 μm and measure the potential there twice.
10. Move back to center of wrinkle and make two measurements
11. Move 500 μm in the opposite direction and measure the surface potential there twice.
12. Move 500 μm more away from the wrinkle and measure the surface potential there twice.

The protocol required two types of the measurements of wrinkles of the human forehead: non-contact Dermacorder® and surface contact probe.

### Dermacorder®

The Dermacorder® is a non-contact electric field imaging device that can detect the electric field inside the epidermis, even though the probe is outside the skin. The operating principal of the Dermacorder® is similar to a parallel plate capacitor, where the two plates are connected by a conductor, and where the distance between the plates oscillates. One plate has an unknown voltage and the other plate has a known voltage that can be varied. In general, as the distance between the plates changes, so too does the capacitance. Now, if the known voltage is varied, we can find a voltage at which the capacitance becomes zero, even though the plate separation is still changing. That applied voltage for which the capacitance is zero and no longer oscillates, is equal to the unknown voltage on the other plate. Subsequent developments led to devices for measuring bioelectric fields wherein the epidermis acts as the surface of unknown voltage and a vibrating probe acts as the surface of known voltage. It is possible to apply known voltages to the probe or the skin, to quickly determine the voltage at which the capacitance is zero and no longer oscillates. That value is the surface potential of the skin. Methods have been developed wherein the two surfaces (skin and probe) are not even joined by a conductor. The Dermacorder® is this type of device.

With the Dermacorder®, a probe is vibrated in the air, close to the skin. Simultaneously, a known voltage, V(b), is superimposed onto the skin. Since the capacitance between two flat conductors is inversely proportional to the distance between them, vibrating the Dermacorder® probe generates an oscillating capacitance that results in an oscillating charge movement on the probe. This is converted into an oscillating voltage signal, and the peak-to-peak value of this output voltage signal is determined by root mean square integration. Since the oscillating charge on the probe is proportional to the voltage difference between the probe and the skin, one could determine the unknown skin surface potential by imposing several different voltage values on the probe or the skin and determining that value for which the charge oscillations go to zero. That voltage must be equal to the skin surface potential. However, rather than stepping through many different voltage values to find the zero point, we determined the peak-to-peak voltages when +10 volts and −10 volts were applied to the skin. These peak-to-peak voltages were plotted (on the ordinate) against the applied voltages (on the abscissa). By extrapolating between the points, the Dermacorder® software is able to calculate the voltage that is equal and opposite to the endogenous skin potential. When a line is drawn between these points, the skin surface potential occurs where the line crosses the abscissa (i.e. where the peak-to-peak voltage equals zero). The slope of this line is inversely proportional to the distance between the probe and the skin. The Dermacorder® software uses that information to maintain a constant distance between the two surfaces, via feed back to one or more z-axis stepper motors.

In the present study, the Dermacorder® probe was a flat gold disc 500 μm in diameter. For greater resolution, this diameter may be reduced. The direction of vibration was perpendicular to surface and the frequency was 1.2 kHz. This frequency may be varied by a person skilled in the art, for greater resolution. During measurement, the closest approach of the probe to a skin surface was about 100 μm, with a total displacement of 30 μm. Total displacements up to at least 50 μm may be useful.

A piezoelectric disk was used to vibrate the probe in the vertical plane. Two stepper motors move the probe in one direction while maintaining a constant distance between the probe and the skin surface via feedback to a z axis stepper motor. The probe was connected directly to the negative input of an Analog Devices 8601 operational amplifier with a 107 ohm feedback resistor. This assembly is housed in plastic in such a way that only the flat probe is exposed, while the plastic is coated with silver paint that is grounded to act as a shield. Dermacorder® software, written in C++, plotted the surface potential as the probe scans over the surface of the skin in 500 μm steps.

### Surface Contact Probe

The second approach directly contacts the stratum corneum to measure the surface potential using a surface contact probe attached to a very high input impedance op amp through an active low pass filter. The probe contacts the surface of the skin in the center of a wrinkle and on either side of the wrinkle, to detect voltage differences generated by the current flowing beneath the stratum corneum. A unity gain op amp with an input resistance of 1012 ohms connected through an active Sallen and Key low pass filter with a cut-off frequency of 8 Hz, was used. The output voltage was displayed on an Agilent 34405A digital multimeter and recorded into an Excel file on a computer.

We began using a gold-plated, spring-loaded contact probe but this did not provide reproducible measurements probably due to electrode polarization. Reproducible readings were obtained with a sintered Ag/AgCl electrode combined with a conductive saline gel coating (Signa Gel, Parker Laboratories). This probe was then used.

The surface probe was mounted to an X-Y-Z micro-positioner so that the probe could be precisely centered in each wrinkle and at 0.5 mm steps on either side.

### Measurements of Endogenous Electric Field of Wrinkles

TABLE 1
Endogenous Electric Field Values of Wrinkles (mV/mm)

| Subject | Wrinkle | Dermacorder ® | Surface probe |
|---|---|---|---|
| 1 (61 y. old) | 1 | −340 | 14 |
| male | 2 | −270 | −40 |
| | 3 | −700 | 0 |
| 2 (55 y. old) | 1 | −210 | 6 |
| male | 2 | −345 | 14 |
| | 3 | −150 | −2 |
| 3 (53 y. old) | 1 | −290 | 0 |
| male | 2 | −440 | −20 |
| | 3 | −100 | −57 |
| 4 (59 y. old) | 1 | −75 | 14 |
| male | 2 | −165 | 20 |
| | 3 | −240 | −30 |
| 5 (63 y. old) | 1 | −430 | −40 |
| male | 2 | −400 | 30 |
| | 3 | −320 | 15 |
| Average | | −280 ± 41 | |

The Dermacorder® detected a mean electric field of −280±41 mV/mm from 15 wrinkles in 5 males 53-63 years of age. The center of the wrinkle was always negative with respect to either side.

According to the methodology described above, measurements were also made of a single female subject (55 y. old). Wrinkles measured in this subject averaged −560 mV/mm, compared to −280 mV/mm for the men.

For the male subjects, the sintered Ag/AgCl electrode detected a mean field on the surface of the stratum corneum of 14±7 mV/mm. The direction of the field, as detected by this method was variable. In 6 out of 15 wrinkles, the center of the wrinkle was measured to be more negative than the sides of the wrinkle.

### Discussion

Not surprisingly, the non-contact Dermacorder® gave more reliable results than the surface contact probe. Although both were used to measure, for the first time, an electric field endogenous to a wrinkle, the Dermacorder® was developed, in part, to overcome the shortcomings of the surface contact probe, which include invasiveness and noise distortion. Movement of the test subject is also more of a negative factor with the surface probe, which required about 5 minutes to complete a set of readings, compared to about 45 seconds for the Dermacorder®.

Measurements on 15 wrinkles from five male volunteers indicated the presence of a strong lateral electric field propagating along the upper epidermis. The average magnitude of this electric field is −280±41 mV/mm, which is about three times larger than characteristic electric fields generated from skin wounds. A recent study using the Dermacorder demonstrated that values for electric field strengths of human wounds is about 80 mV/mm (Nuccitelli, 2008). Furthermore, the wrinkle electric field and the wound electric field in human skin, have the same polarity, being more negative at deeper layers, both of which are opposite to undamaged, unwrinkled skin.

In the field of electrotherapy, electric currents externally applied to the skin have been used to treat various sorts of skin lesions. The applied electric current enhances the curative processes of the endogenous wound current. Most often, electric currents have been applied to the skin via an electrical apparatus. However, topical preparations that create a microcurrent when applied to the skin, are also known. One example of topically applied microcurrent for the treatment of skin lesions is U.S. Pat. No. 6,306,384. While the reference does mention the use of externally applied microcurrent to promote healthy skin and to treat the irritation associated with wounds or dry skin, the ability of externally applied microcurrent to treat or diagnose wrinkles is not suggested.

As note above, it is known that as a wound heals, the wound current and wound electric field return to a normal transepidermal electric field. This is true, whether the wound heals on its own or is aided by an externally applied electric field. The skin electric field returns to pre-wound magnitude and pre-wound polarization.

### Conclusions

Our observations, suggest for the first time, that skin wrinkles associated with ageing, can be characterized by an endogenous wrinkle electric field, whose existence we have postulated and demonstrated. The endogenous wrinkle electric field can be measured by the methods described herein, and no doubt, by other methods, currently existing or yet to be developed. Not surprisingly, the non-contact Dermacorder® gave more reliable results than the surface contact probe, although both were used to measure, for the first time, an electric field endogenous to a wrinkle.

Moreover, the course of wrinkle progression can be characterized by changes in the wrinkle electric field. Thus, we disclose a new kind of wrinkle diagnosis, associated with the endogenous wrinkle electric field. Also, we disclose a new kind of treatment assessment, associated with the endogenous wrinkle electric field. The treatments that may be assessed by our new methods, may be treatments that affect a wrinkle by directly manipulating the wrinkle electric field or treatments that directly address some other feature of a wrinkle, for example, collagen enhancement treatments. Either way, the results of that treatment will manifest as measurable changes in the endogenous wrinkle electric field.

A main object of the invention is to provide methods of characterizing the electric field that arises specifically from a skin wrinkle. Such a method comprises the steps of measuring the endogenous wrinkle electric field, noting the intensity and polarity of the electric potential that arises from the wrinkle; associating a more negative reading with a more severe wrinkle or a less negative reading with a less severe wrinkle.

Another object of the invention is to provide methods of treating a wrinkle, based on electric field properties wrinkles. Such a method comprises: applying to a wrinkle, a treatment that tends to reverse the electric field polarity of the wrinkle.

Another object of the invention is to provide methods of preventing a wrinkle, based on electric field properties wrinkles. Such a method comprises: identifying a section of skin for protective treatment, applying to the section, a treatment that causes the section of skin to retain its electric field polarity.

Another object of the invention is to provide methods of characterizing the progression of a skin wrinkle. Such a method comprises the steps of: making a first measurement of the endogenous wrinkle electric field; thereafter, waiting an amount of time with or without treating the wrinkle; making a second measurement of the endogenous wrinkle electric field; and comparing the two measurements for changes in intensity and/or polarity.

Another object of the invention is to provide methods of evaluating the efficacy of a skin wrinkle treatment, based on the electric field properties wrinkles. Such a method comprises the steps of: making a first measurement of the endogenous wrinkle electric field; thereafter, applying a treatment to the wrinkle; thereafter, making a second measurement of the endogenous wrinkle electric field; and comparing the two measurements for changes in intensity and/or polarity.

Another object of the invention is to provide methods of comparing the efficacy of two skin wrinkle treatments, based on the electric field properties wrinkles. A more effective wrinkle treatment is one for which the second measurement minus the first is larger. For example, if a first measurement before treatment is −280 mV/mm and a second measurement after treatment is −200 mV/mm, an improvement in the wrinkle is indicated by the difference −200—(−280)=+80. If a different treatment is tried, and the before (first) and after (second) measurements are −250 mV/mm and −150 mV/mm, respectively, then the improvement in wrinkle is indicated by −150−(−250)=+100. The second treatment produced a larger positive change in the endogenous wrinkle electric field, which indicates a more efficacious treatment. Thus, a method of comparing the efficacy of two skin wrinkle treatments comprises the steps of: selecting a first wrinkle and a second wrinkle; making a first measurement of the endogenous wrinkle electric field of the first wrinkle; thereafter, applying a first treatment to the first wrinkle; thereafter, making a second measurement of the endogenous wrinkle electric field of the first wrinkle; subtracting the first measurement from the second for the first wrinkle; making a first measurement of the endogenous wrinkle electric field of the second wrinkle; thereafter, applying a first treatment to the second wrinkle; thereafter, making a second measurement of the endogenous wrinkle electric field of the second wrinkle; subtracting the first measurement from the second for the second wrinkle; and comparing the differences for the first and second wrinkle.

Another object of the invention is to provide methods of formulating topical wrinkle treatment products, based on the electric field properties wrinkles. In one type of topical wrinkle product, the product superimposes an electric field on to the endogenous electric field of the wrinkle. Preferably, a superimposed electric field has a polarity that is opposite to that of the endogenous electric field. It may also be preferable if the net electric field that results from the superposition of the endogenous electric field and the applied electric field of the topical composition, is substantially similar to the transepidermal electric field of healthy, non-wrinkled skin. Thus, one method of formulating topical wrinkle treatment products, based on the electric field properties wrinkles, comprises the steps of: formulating an electric field-generating wrinkle product; selecting a wrinkle; making a first measurement of the endogenous wrinkle electric field of the wrinkle; thereafter, applying the electric field-generating product to the wrinkle; thereafter, making a second measurement of the endogenous wrinkle electric field of the wrinkle; subtracting the first measurement from the second measurement; based on the difference of the first and second measurement, reformulating the wrinkle product so that the generated electric field superimposed on the endogenous electric field of the wrinkle is substantially similar to the transepidermal electric field of healthy, non-wrinkled skin. These steps may be repeated. Also, the step of making a second measurement may be performed while the applied product is still actively generating an electric field, (which may be within seconds or minutes of application). Or, the second measurement may done at a time when the applied product is no longer active on the skin, perhaps, hours or days later. In the first case, the second measurement would be of a net electric field (wrinkle plus product) in the vicinity of the wrinkle. In the second case, the second measurement would be of an endogenous wrinkle electric field which resulted from treatment with the product.

In another type of topical wrinkle product, the product alters the lateral electric current in the vicinity of the wrinkle. In general, the topical wrinkle product may increase, decrease and/or change the direction of the lateral current. Preferably, the change in current results in a net, lateral electric current of zero, in the vicinity of the wrinkle. Thus, another method of formulating topical wrinkle treatment products, based on the electric field properties wrinkles, comprises the steps of: formulating a wrinkle product; selecting a wrinkle; making a first measurement of the endogenous lateral electric field of the wrinkle; thereafter, applying the product to the wrinkle; thereafter, making a second measurement of the lateral electric field in the vicinity of the wrinkle; if the net lateral electric field is not zero, reformulating the wrinkle product so that the net, lateral electric field in the vicinity of the wrinkle is closer to zero. These steps may be repeated. Also, the step of making a second measurement may be performed while the applied product is still actively altering the lateral electric current, (which may be within seconds or minutes of application). Or, the second measurement may done at a time when the applied product is no longer active on the skin, perhaps, hours or days later. In the first case, the second measurement would be of a net lateral electric field (wrinkle plus product) in the vicinity of the wrinkle. In the second case, the second measurement would be of an endogenous wrinkle electric field which resulted from treatment with the product.

What is claimed is:

1. A method of characterizing the electric field that arises specifically from a skin wrinkle, comprising:
measuring the endogenous wrinkle electric field;
noting the intensity and/or polarity of the electric potential that arises from the wrinkle; and
associating a more negative reading with a more severe wrinkle or a less negative reading with a less severe wrinkle.

2. The method of claim 1 wherein a vibrating probe device is used to measure the endogenous wrinkle electric field.

3. The method of claim 2 wherein the vibrating probe device is a non-contact device.

4. the method of claim 3 wherein the wrinkle is scanned with the vibrating probe device at the wrinkle center and at locations within about 5 mm on either side thereof.

5. The method of claim 3 wherein a vibrating probe is s a flat gold disc, not larger than 500 μm in diameter.

6. The method of claim 3 wherein a vibrating probe vibrates perpendicular to the skin surface and has a total displacement of not more than about 50 μm.

7. The method of claim 1 wherein a surface probe device is used to measure the endogenous wrinkle electric field.

8. A method of treating wrinkles, based on electric field properties of wrinkles, comprising applying to a wrinkle, a treatment that tends to reverse the electric field polarity of the wrinkle.

9. A method of preventing wrinkles, based on electric field properties of wrinkles, comprising:
identifying a section of skin in need of protective treatment; and
applying to the section, a treatment that causes the section of skin to retain its electric field polarity.

10. A method of characterizing the progression of a skin wrinkle, based on electric field properties of wrinkles, comprising:
making a first measurement of the endogenous wrinkle electric field;
thereafter, waiting an amount of time with or without treating the wrinkle;
making a second measurement of the endogenous wrinkle electric field; and
comparing the two measurements for changes in intensity and/or polarity.

11. A method of evaluating the efficacy of a skin wrinkle treatment, based on the electric field properties wrinkles, comprising:
identifying a wrinkle for treatment;
making a first measurement of the endogenous wrinkle electric field;
thereafter, applying a treatment to the wrinkle;
thereafter, making a second measurement of the endogenous wrinkle electric field; and
comparing the two measurements for changes in intensity and/or polarity.

12. A method of comparing the efficacy of two skin wrinkle treatments, based on the electric field properties wrinkles, comprising:
selecting a first wrinkle and a second wrinkle;
making a first measurement of the endogenous wrinkle electric field of the first wrinkle;
thereafter, applying a first treatment to the first wrinkle;
thereafter, making a second measurement of the endogenous wrinkle electric field of the first wrinkle;
subtracting the first measurement from the second for the first wrinkle;
making a first measurement of the endogenous wrinkle electric field of the second wrinkle;
thereafter, applying a first treatment to the second wrinkle;
thereafter, making a second measurement of the endogenous wrinkle electric field of the second wrinkle;
subtracting the first measurement from the second for the second wrinkle; and
comparing the differences for the first and second wrinkle.

13. A method of formulating topical wrinkle treatment products, based on the electric field properties wrinkles, comprising:
formulating an electric field-generating wrinkle product;
selecting a wrinkle;
making a first measurement of the endogenous wrinkle electric field;
thereafter, applying the electric field-generating product to the wrinkle;
thereafter, making a second measurement of the endogenous wrinkle electric field;
if the endogenous electric field is not substantially similar to the transepidermal electric field of healthy, non-wrinkled skin, reformulating the wrinkle product so that the endogenous electric field of the wrinkle is closer to the transepidermal electric field of healthy, non-wrinkled skin.

14. The method of claim 13 wherein the step of reformulating is repeated at least once.

15. The method of claim 13 wherein the second measurement is made while the product is still active on the skin.

16. The method of claim 13 wherein the second measurement is made when the product is no longer active on the skin.

17. A method of formulating topical wrinkle treatment products, based on the electric field properties wrinkles, comprising:
formulating a wrinkle product;
selecting a wrinkle;
making a first measurement of the lateral electric field in the vicinity of the wrinkle;
thereafter, applying the product to the wrinkle;
thereafter, making a second measurement of the lateral electric field in the vicinity of the wrinkle;
if the lateral electric field is not zero, reformulating the wrinkle product so that the lateral electric field in the vicinity of the wrinkle is closer to zero.

18. The method of claim 17 wherein the step of reformulating is repeated at least once.

19. The method of claim 17 wherein the second measurement is made while the product is still active on the skin.

20. The method of claim 17 wherein the second measurement is made when the product is no longer active on the skin.
